(19) United States
(12) Patent Application Publication
(10) Pub. No.: US 2004/0186328 A1
(43) Pub. Date: Sep. 23, 2004

(54) Process of heterocoupling by electrolytic microbattery, use of cobalt for implementing said coupling and composition for doing so

(76) Inventors: Jacques Perichon, Savigny-sur-Orge (FR); Corinne Gosmini, Savigny-sur-Orge (FR); Mylene Bastienne, Fleury Merogis (FR); Paulo Gomes, Le Perreux (FR)

Correspondence Address: BURNS DOANE SWECKER & MATHIS L L P, POST OFFICE BOX 1404, ALEXANDRIA, VA, 22313-1404, US

(21) Appl. No.: 10/743,137

(22) Filed: Dec. 23, 2003

(30) Foreign Application Priority Data
Dec. 23, 2002 (FR) 02/16525

(51) Int. Cl. C07C 17/26; C07C 17/38

(52) U.S. Cl. 570/237

(57) ABSTRACT

This invention has as its object a process for preparation of vinyl aryl derivatives by an electrochemical path.

This process is defined in that it consists in subjecting a composition that comprises a cobalt salt, an aromatic halide and a vinyl ester to the action of a metal that is at least as reducing as zinc.

Application to organic synthesis.

This invention has as its object a process of synthesis of a vinyl or allyl aryl compound starting from a reaction of heterogeneous coupling by a means of the electrolytic type between aryl compounds and vinyl compounds or allyl compounds.

It aims more specifically at the use of cobalt salts, especially cobalt(II) salts, as electrochemical coupling catalysts between an aryl derivative and a vinyl derivative in the presence of a metal in the metallic or elementary state.

As for the coupling between vinyl derivatives and aryl derivatives to yield vinyl aryl derivatives, there is almost no description of it. The only document relating to it is the report published in Tetrahedron, Volume 48, No. 4, pages 719 to 726, where a reaction of this type is described in the presence of palladium(II) salts that are supported on silylated montmorillonite. No mechanism is proposed, and the absence of elements described above does not make it possible to propose even one balanced reaction equation because as soon as they are solved, all of the proposed reaction equations lead to apories.

These reactions would, however, allow easy and particularly advantageous access to useful, complex derivatives, especially in the fields of pharmacy and agrochemistry.

This is why one of the objects of this invention is to provide a coupling process between vinyl derivatives and aryl derivatives that does not require the use of expensive catalysts. Another object of this invention is to provide a process of the preceding type that furnishes good yields.

Another object of this invention is to provide a process of the preceding type that is doubly ipso, the bond between the vinyl molecule and the aryl molecule being accomplished at the site where the two leaving groups were found.

Another object of this invention is to provide a process that yields few parasitic reactions, especially a reaction that yields little symmetrical coupling and that yields few reductions to lead to hydrogenated compounds in place of the leaving groups.

As for the coupling between allyl derivatives and aryl derivatives to yield allyl aryl derivatives, there is almost no description of it.

These reactions would allow, however, easy and especially advantageous access to useful, complex derivatives, especially in the fields of pharmacy and agrochemistry.

This is why another object of this invention is to provide a process of coupling between allyl derivatives and aryl derivatives that does not require the use of expensive catalysts. Another object of this invention is to provide a process of the preceding type that furnishes good yields.

Another object of this invention is to provide a process of the preceding type that is doubly ipso (here, the ablative of the Latin “ipse” is used to indicate that functionalization is done on the same carbon as the one that the leaving group was carrying), the bond between the molecule that carries the unsaturation, especially allyl, and even homoallyl, and the aryl molecule being accomplished at the site where the two leaving groups were found.

Another object of this invention is to provide a process that yields few parasitic reactions, especially a reaction that yields little symmetrical coupling and that yields few reductions to lead to hydrogenated compounds in place of the leaving groups.

Another object of this invention is to provide a reaction that can be easily carried out on a large scale and that does not require the use of electrical current nor an electrolytic technique per se.

These objects and others that will appear below are achieved by means of a process of heterocoupling, between an aryl (pseudo)halide and an ethylene unsaturation carrier and a leaving group, advantageously an ester, and even an ether, especially of allyl and vinyl, which consists in subjecting the two substrates to a reduction by means of a metal in the elementary state in the presence of cobalt (II).

Although one possible explanation for this reaction is a reaction involving a microbattery on a particle of a reducing metal, it takes place without delivery of external current, and this therefore cannot be an electrolytic reaction. With consideration of the aforementioned, the operating conditions are considered to be non-electrolytic.

The metal in the elementary state can be an alloy provided that the alloy is sufficiently electroreducing.

The redox potential of said metal is such that the latter is as much as or more reducing than zinc.

It is not always easy to find redox potential tables for a certain nonaqueous solvent. It should also be mentioned that in the first approximation for the order of the reducing capacity of metals and alloys, it will be possible to refer to scales of reducing capacity that are established based on the values of the standard reduction potential. (Refer to the various editions of the “Handbook of Chemistry and Physics” for these scales.)

The redox potential of zinc is given as 0.76 volt [more specifically −0.7628 volt for the reduction reaction of the zinc ion (Zn⁺⁺+2 e⁻->Zn^{o})] relative to the hydrogen electrode. Metals and alloys that are more reducing than hydrogen and whose absolute value potential (measured in water) is at least equal to 0.7 will be considered as entering into the field of the invention.

Metals and alloys that are more reducing than zinc have a potential that is at least equal to 0.8 volt in absolute value.

The zinc itself can be used especially for coupling with the allyl derivatives, but, especially for coupling with the vinyl derivatives, it is preferable to use more reducing metals (absolute value potential at least equal to 0.8, advantageously 0.9, and preferably 1 V). When they are more reducing than manganese (˜|1|V), these metals or alloys are advantageously used in the presence of manganese salts (Mn⁺⁺), the latter being advantageously at a concentration that is selected in the same concentration range as that defined below for cobalt, preferably in a molar quantity that is at least equal to that of the cobalt (II) salts.

Highly electroreducing metals like magnesium (˜|2.375| V) and aluminum should be avoided only; the presence of the manganese ion reduces their drawbacks; it is still preferable to avoid them as well as the other highly reducing metals (i.e., at least as reducing as at least one of these elements) such as alkalines and other alkaline-earths.

Manganese can be used as a metal, in which case the metal as it dissolves during the reaction will guarantee good stability of the potential in the vicinity of the metallic surface, and it will be of little advantage to add manganese ions to the reaction mixture.

Thus, when the metal is selected from among metals (or alloys) that are more electroreducing than manganese, it is advantageous that the medium contain manganese ions, advantageously at a concentration of between 2×10⁻³ and 10⁻¹ M (including the limits).

The amount of metal to be used is advantageously at least equal to the stoichiometric amount, preferably at least 1.5 times and even 2 times. The metal is advantageously in a divided form of granulated turnings and primarily powder.

It is preferable to provide a reagent that is reported to depassivate metal (iodine or acid, see below).

According to another aspect of the invention, it has also been shown that the presence of acid, advantageously organosoluble, made it possible to significantly improve yields.

These acids can be especially carboxylic acids, fatty acids or halogenated acids, and even perhalogenated acids. The perfluorinated acids exhibit a special advantage due to their solubility in the organic phases and their relatively strong acidity.

If acidity is needed, it is a good idea to control the amount using the acids that were mentioned above, and especially carboxylic acids such as trifluoroacetic acid and acetic acid itself. Fatty acids can also be used whether they are perhalogenated (generally perfluorinated) or not on the carbon that carries the carboxylic function.

The concentration of acid is advantageously at least equal to 10⁻³ M, advantageously 5×10-3 M. The higher value is essentially limited by the amount of metal or the metal alloy in the medium. The amount of acid should be metal or alloy, less than that which is necessary to dissolve all of the metal or the metal alloy and to leave enough metal or metal alloy for the reaction.

According to this invention, it has been possible to replace equivalent amounts (expressed in moles) thereof by iodine (12).

Generally, whether iodine or acids, it is preferable to remain at a relatively weak level to avoid parasitic reactions, especially the formation of hydrocarbon in place of the desired coupling product. Generally, amounts of acid or iodine that are less than 10% and of the aromatic compound comprising one of the precursor substrates of the coupling product expressed in mols, advantageously at most equal to 5%, are also used.

Although the form of the cobalt in the vicinity of the metal in the elementary state had not been completely explained, it was possible to show that the presence of cobalt coordinating agents proved important and made it possible to modify the yields very significantly. It could be that this presence may have a role in the optional coordination between the ethylenic unsaturation and the cobalt.

Remembering that an ester is defined as the product of condensation between a carrier of a hydroxyl function and a Bronstedt acid, i.e., a carrier of an acidic hydrogen with elimination of water, it is deduced therefrom that the intended esters can be halides (mainly heavier than the fluorides), sulfates, sulfonates, phosphates, phosphonates and carboxylates; the latter are preferred.

Unsaturation is advantageously near the atom that was the carrier of the acidic hydrogen, i.e., said carrier atom is advantageously in vinyl-position (L is then not a single bond), allyl-position (L is a methylene link, optionally substituted) or even homoallyl-position (L is an ethylene link, optionally substituted); preferably in the vinyl- or allyl-position.

The reaction can be written roughly as follows:

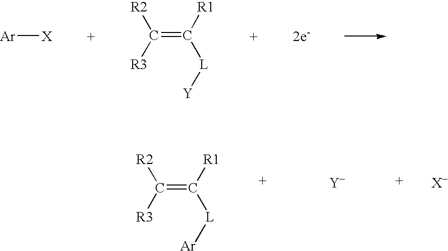

The electrons are provided here by the metal or alloy that is used.

Formulas in which L represents a divalent arm ensuring the link between the vinylic unsaturation and the remainder of the acid (Y is noted here) after having ignored hydrogen. Thus, when the ester is vinyl, L is absent, -L- symbolizing then the single bond linking Y and the vinylic unsaturation. When -L-represents something other than a single bond, L is advantageously an alkylene group; preferably ethylene or methylene, more preferably methylene. In the latter case, the ester is allylic.

Formulas in which Y corresponds to a leaving group that can exist in the form Y⁻¹ such as phenate, and even alcoholate, but advantageously selected from among the halogens and the carboxylates lato sensu and the pseudohalogens, it is desirable that it is such that the YH acid has a pKa (measured in water) that is at most equal to 9, advantageously 7, and preferably 5. It is advantageously selected from among the halogens and carboxylates lato sensu and the (pseudo)halogens.

The case where Y is such that it forms an ether with the compounds that carry an ethylene double bond is hardly to be considered except when aryl-allyl coupling is intended.

A pseudohalogen is defined as a group whose removal leads to an oxidized anion, the anionic charge being carried by the chalcogen atom (generally oxygen) and whose acidity is at least equal to that of acetic acid, advantageously to the second acidity of sulfuric acid and preferably to that of the trifluoroacetic acid. To be located on the scale of acidities, it is a good idea to refer to the pKa for medium to strong acidities starting with carboxylic acids to acetic acid and to be located on the scale of Hammett constants starting from trifluoroacetic acid.

Carboxylate lato sensu is defined as any radical such that its anionic form comprises the atomic sequence—CO—O⁻; thus not only are the carboxylate functions being linked to one carbon atom (the latter are, however, preferred) intended, but also carbamic acids and alkyl carbonates. The most commonly used are the aryl radical acids (such as benzoic acid) or the aliphatic radical acids (i.e., whose carboxylic function is carried by a hybridization carbon sp³, advantageously which carbon is itself linked only to carbons or hydrogens as is the case in alkanoic acids). It should be noted that the acid function can be provided by the carrier of an unsaturation and thus can form a cyclic derivative such as vinyl or allyl lactones.

It is preferable that the leaving groups (Y or X), when they are organic, have at most 10 carbon atoms, preferably at most 4.

If it is desired to avoid all parasitic reactions, it is preferable to avoid the substituents comprising reactive hydrogens such as hydrogens on the amides (that are therefore advantageously protected or peralkylated) or on an oxygen.

Formulas in which R₁, R₂ and R₃, which may or may not be different, are selected from among hydrogen, the functions that are more difficult to reduce than the function Y, and among the hydrocarbon radicals in particular alkyls and aryls.

Thus, among the functions that are more difficult to reduce than Y, it is possible to cite, when Y forms an ester, the ether functions, the carboxylic functions (linked or not to the remainder of the molecule by the carbon), the functions among which Y is selected provided that these functions are less reducible than Y. The order of reducibility can be easily determined under operating conditions by routine experiments. Purely by way of indication, it can be noted that regarding the halides, the higher the atomic number, the more the halide is reducible and more generally (and more-roughly) the stronger the acid is which corresponds to the leaving group, the more the ester of the corresponding vinyl is reducible (but it should be noted that the anions can themselves be reduced and cause parasitic reactions).

Among the groups that are relatively reducible and that should be emphasized, it is possible to cite the perfluorinated groups: one of the solutions is to influence the current density and therefore here the potential of the metal or alloy that is used.

The hydrocarbon radicals are preferably either of an aromatic nature or an aliphatic nature, i.e., the carbon that ensures the link to the remainder of the molecule is hybridization carbon sp³; these aliphatic radicals are in general alkyls (alkyl is used in the etymological meaning of an alcohol from which the OH function is removed), including aralkyls. It should be pointed out that the hydrocarbon radicals that have a double bond conjugated with the double bond yield only very mediocre results.

To be effective, it is desirable that the cobalt be present at a minimum concentration of at least 10⁻³ M.

To be economical, it is preferable that the cobalt not be overly concentrated, rather it is preferred that the cobalt content be at most equal to 0.2 M.

The reaction medium advantageously comprises a solvent, and this solvent should be polar enough to dissolve metals or more exactly the salts of the metals used, and it should be lipophilic enough to dissolve, at least partially, the substrates from which it is desired to form the vinyl aryl.

It is preferable to use solvents with such low acidity that the reactions with hydrogen are as little pronounced as possible. Thus, the primary alcohols are too acidic.

More specifically, said polar aprotic solvents such as the following, for example alone or in a mixture, will be preferred:

Purely oxidized solvents, in particular ethers, preferably polyethers such as dimethoxy-1,2-ethane or cyclic ethers such as THF or dioxane;

amides or ureas (DMF, N-methylpyrrolidone-2, imidazolidone, tetramethyl urea, dimethoxypropylene-urea, etc.);

sulfones (for example sulfolane) or sulfoxides (such as DMSO);

and, to the extent that they are liquid under operating conditions, nitrogen-containing derivatives, nitrogen-containing heterocyclic compounds, especially pyridine, and compounds with a nitrile function (for those that are preferred, see below);

and, to the extent that they are liquid under operating conditions, complexing agents (crown ether, HMPT, tris-(dioxa-3.6-heptyl)amine (TDA-1)) that improve the smooth running of the reaction by increasing conductivity, increasing the reactivity of the anion, and preventing metal deposits on the cathode.

Without this explanation being limiting, it would seem that these advantageous phenomena are correlated with the capacity to complex the metallic cations or in a mixture.

As indicated above, the solvents that are used can themselves play the part of complexing agents or coordinating agents. They can especially, and this is advantageous, have one or more of the functions of coordination mentioned above.

The solvent can be a mixture of an apolar solvent and a polar solvent as defined above by the donor index.

To facilitate the separation of the products from the reaction media, it is preferable that said solvent has a boiling point that is significantly different from that of the compound to be synthesized and the starting compound.

Ordinarily, to facilitate the reaction and improve the conductivity of the medium, in general saline electrolytes are used, sometimes called bottom salts, optionally modified by the presence of complexing agents. These electrolytes are selected such that they do not disturb the reactions on the anode and cathode. The latter is advantageously inert. In this invention, however, taking into account that the reaction seems to proceed by the formation of a microscopic battery in the vicinity of the surface of the metal, the presence of the bottom salt does not yield significant advantages while making the treatment of the reaction mixture more cumbersome.

According to one of the most economical implementations of this invention, the salts that are formed by the dissolution of the metals that are used are used as a bottom salt.

The anions introduced into the reaction mixture (especially with the cobalt or manganese salts) can be the anions that are usual for indifferent electrolytes, but it is preferable that they be selected from among those that are released by the reaction, essentially halides, or, for example, by complex anions of type BF₄⁻, PF₆₋, or ClO₄⁻. Among the preferred anions, it is possible to cite those that are obtained from fluorinated acids or their imides (TFSI, triflates, etc.). By way of indication, it should be pointed out that DMF, used with tetrabutylammonium tetrafluoroborate as a bottom salt at the concentration of 0.01 M, yielded good results.

Said reaction can be conducted at many temperatures, but it is preferable to conduct this electrolysis at a temperature that is at most equal to 100° C. and at most equal to the boiling point of the solvent.

An interval yielding good results is the interval between 0 and 50° C.; it is a closed interval, i.e., including the limits.

Pressure is of little importance to the reaction, except if one of the reagents or the solvent has an especially low boiling point.

For practical reasons, however, the pressure is preferably the atmospheric pressure of the location in question.

In the aforementioned case where one of the components of the reaction medium is especially volatile and where it is desired to keep this component in the reaction medium, it is then possible to increase the pressure; this pressure is generally then an autogenous pressure resulting from the reaction in a closed chamber.

The aryl substrates (Ar—X) that can be coupled to the compounds that carry an ethylenic unsaturation according to this invention represent a wide range of compounds. The halides are generally halides corresponding to relatively heavy halogens, i.e., halogens that are heavier than fluorine (iodide, bromide, chloride).

It can also be indicated that when the halogen is linked to an aromatic core that is low in electrons, it is preferable to use bromines or chlorines as halogen, the chlorines being reserved for cores that are especially low in electrons. The condition is almost always met by heterocyclic compounds with six chain links, but in the case of homocyclic aryl hexacyclic substrates, to use a chloride, it is preferable that the sum of the Hammett constants ₚ (sigma p) of the substituents (not taking into account the starting halide) is at least equal to 0.4, preferably 0.5. By contrast, the cores that are especially high in electrons can be used as a halide or iodide.

The best results are those that are obtained starting from chlorinated aromatic compounds (X is chlorine) that are stripped of electrons.

For more details on Hammett constants, it is possible, for example, to refer to the third edition of the manual written by Professor Jerry March “Advanced Organic Chemistry” (pages 242 to 250) and edited by John Wiley and Sons.

The heterocyclic compounds with five chain links and that comprise as heteroatom a chalcogen (such as furan and thiophene) also yield acceptable results.

As was mentioned above, the reduction in electrons from the core can be caused either by the presence of electroattractor groups as substitutents (especially those that are designated by R, R₁′, R₂′, and R₃′ in the formulas provided below), or, in the case of cores with six chain links, by the replacement of a carbon by a heteroatom. In other words, the core that is reduced in electrons can be a heterocyclic core with six chain links, especially the heterocyclic cores that have an atom from the column of nitrogen and more particularly the nitrogen.

Among the electroattractor groups (especially those that can be designated R, R₂′, R₂′, or R₃′ in the formulas provided below) leading to good results, it is suitable to cite acyl groups, nitrile groups, sulfone groups, carboxylate groups, trifluoromethyl groups or more generally perfluoroalkyl groups and halogens of a lower order than halide, which will be replaced by the allyl radical.

Among the donor groups (especially those that can be designated by R, R₁, R₂′, or R₃′ in the formulas provided below), i.e., yielding mediocre results with chlorine, but good results with bromine, it is possible to cite alkyloxyl groups, alkyl groups, amine groups and dialkylamine groups.

Aryl substituents (especially those that can be designated by R, R₁′, R₂′, and R₃′ in the formulas provided below) are weakly electroattractive and do not significantly change the properties of the aryl radical.

The aromatic derivative substrate of this process corresponds advantageously to the following formula:

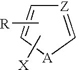

where:

Z represents a trivalent chain link that is selected from among a link of formula

—C(R₁)=, and an atom of column V, advantageously a nitrogen;

R represents any optional substituent whose values are presented in detail below;

X represents halogen, and even a starting pseudohalogen (provided that its acidity on the Hammett scale is at least equal to that of chlorine);

A represents either a divalent link that is selected from among the ZH groups or from among the chalcogens that are advantageously of an order that is at least equal to that of sulfur, or from among the unsaturated divalent groups with two chain links

—CR₂═CR₃—, N═CR₂—; —CR₂═N—.

To the extent that they are carried by the contiguous atoms, two of the radicals R, R₁′, R₂′, and R₃′ can be linked to form rings.

Thus, the aryls can have especially the formula:

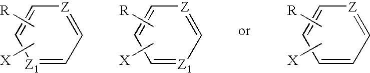

where:

Z₁ is selected from among the same meanings as those provided for Z;

radicals R, R₁₁, R₂′, and R₃′ are selected from among hydrogen and the aforementioned substituents and especially:

electroattractor groups, in particular acyl groups, nitrile groups, sulfone groups, carboxylate groups, trifluoromethyl groups or more generally perfluoroalkyl groups and halogens of a lower order than halide that will be transformed into a coupling product;

donor groups, especially the aryloxyl groups, alkyloxyl groups, the hydrocarbyl groups such as aryl and alkyl (the latter word being used in its etymological meaning), amine groups, including groups that are mono- and disubstituted by alkylamine hydrocarbon groups.

It is desirable that the Ar—X substrates have at most 50 carbon atoms, advantageously at most 30 carbon atoms, and preferably at most 20 carbon atoms. The same is true of the substrates that have said double bond.

The especially advantageous substrates include the halides, preferably aryl chlorides, that carry in particular in meta-position an aliphatic carbon (i.e., sp³) that carries at least two fluorines, for example halides, preferably trifluoromethylaryl chlorides.

It is preferable that the cobalt be coordinated, however, the optimum coordination conditions are a little different for the vinyl esters, on one hand, and for the other esters, especially allyl, on the other hand.

This description now pertains more specifically to the implementation in which the ester is vinyl; in this case, L is absent and therefore -L- is a single bond: the equation above then becomes:

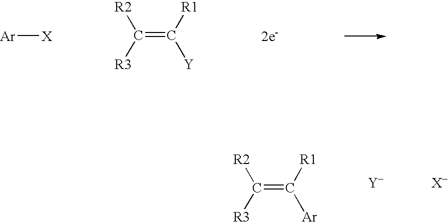

The vinyl seat of the reaction provides only very mediocre results when it is conjugated with an ethylene double bond (and even an acetylene double bond; the negative effect is much less for a conjugation with an aromatic core) to provide a butadiene skeleton.

In general, the number of carbons of the vinyl derivative is less than 50, advantageously 30.

Actually, during studies that led to this invention, it was shown that in the presence of cobalt, the coupling above took place with good yields.

Although the form of the cobalt close to the metal in the elementary state has not been completely clarified, it was possible to show that the presence of cobalt coordinating agents proved important and made it possible to increase the yields very significantly. It could be that this presence may have a role in the optional coordination between ethylenic unsaturation and cobalt.

Although an effect can be demonstrated when solvents that have the property of coordinating the cobalt are used, it is preferable to use specific coordinating agents.

If a return is made to the agents or solvation agents that make it possible to improve the yield significantly, it is possible to indicate that it is possible to use compounds that have a high donor index. More specifically, it is possible to indicate that it is preferable that donor index D of these solvents, or of these solvation agents, be greater than or equal to 10, preferably less than or equal to 30, advantageously between 20 and 30, including the limits. Said donor index corresponds to AH (enthalpy variation) that is expressed in kilocalories of the combination of said polar aprotic solvent or said coordinating agent with antimony pentachloride.

This is described more specifically in the work of Christian Reichardt: “Solvents and Solvent Effects in Organic Chemistry”—VCH, page 19, 1988. On this page is found the definition of the donor index that is expressed in English terms by “donor number.”

The results are better if the atom that coordinates the cobalt in said solvent or solvation agent is an atom from the column of nitrogen, and advantageously the nitrogen itself.

When a specific coordinating agent that does not play the role of solvent is used, it is possible to cite the functions or group of pyridine, nitrile, phosphine, stibine and imine.

To be effective, it is desirable that the cobalt be present at a minimum concentration of at least 10⁻³ M.

To be economical, it is preferable that the cobalt not be too concentrated; it is also preferred that the cobalt content be at most equal to 0.2 M.

The reaction medium advantageously comprises a solvent, and this solvent should be polar enough to dissolve the metals or more exactly the salts of the metals that are used, and it should be lipophilic enough to dissolve, at least partially, the substrates from which it is desired to form the vinyl aryl.

It is preferable to use solvents that are low enough in acid so that the reactions with hydrogen are as little pronounced as possible. Thus, the primary alcohols are too acidic.

More specifically, the so-called polar aprotic solvents, such as the following, for example, alone or in a mixture, will be preferred:

Purely oxidized solvents, in particular ethers, preferably polyethers such as dimethoxy-1,2-ethane or cyclic ethers such as THF or dioxane;

amides or ureas (DMF, N-methylpyrrolidone-2, imidazolidone, tetramethyl urea, dimethoxypropylene-urea, etc.);

sulfones (for example sulfolane) or sulfoxides (such as DMSO);

and, to the extent that they are liquid under the operating conditions, nitrogen-containing derivatives, nitrogen-containing heterocyclic compounds, especially pyridine and compounds with a nitrile function (for those that are preferred, see below);

and, to the extent that they are liquid under the operating conditions, complexing agents (crown ether, HMPT, tris-(dioxa-3.6-heptyl)amine (TDA-1)) that improve the smooth running of the reaction by increasing conductivity, increasing the reactivity of the anion, and preventing metal deposits on the cathode.

Without this explanation being limiting, it would seem that these advantageous phenomena are correlated with the capacity to complex the metallic cations or in a mixture.

As indicated above, the solvents that are used can themselves play the part of complexing agents or coordinating agents. They can especially, and this is advantageous, have one or more of the functions of coordination mentioned above.

The solvent can be a mixture of an apolar solvent and a polar solvent as defined above by the donor index.

When the solvent is not in itself a complexing agent of the cobalt that is strong enough to obtain optimum results, it is then desirable to use one of the complexing agents that is specific for cobalt, advantageously polydentate, most often bidentate. As functions that play the role of teeth, it is suitable to cite the nitrites (preferably aromatic and/or bidentate) or else the pyridines and the derivatives of the pyridine core, such as the quinoline.

The bipyridyls (o, o′), being bidentate, thus also yield very good results as a separate coordinating agent of the solvent. The preferred complexing agents are those that do not carry a charge, primarily negative, on the atom, or the atoms that carry the bond, coordinating the cobalt; it is also preferable that when said complexing agent carries a charge, the latter is located by the shortest path to at least 4 and even advantageously to at least 5 atoms, preferably 6, primarily when said charge is negative. The cyanides thus are not desirable as a complexing agent of the cobalt.

To obtain improved results and yields, it is preferable that the ratio (coordinating agent(s)/cobalt) between coordinating agent(s), expressed in mol for the monodentates and in equivalent terms for the polydentates and the cobalt ions (expressed in mol) be at least equal to 0.5; advantageously 1, preferably 2, and more preferably 4.

To facilitate the separation of the products with the reaction media, it is preferable that said solvent has a boiling point that is essentially different from the compound that is to be synthesized and the starting compound.

Said electrolysis can be conducted at various temperatures, but it is preferable to conduct this reaction at a temperature that is at most equal to 100° C. and at most equal to the boiling point of the solvent.

An interval that provides good results is the interval between 0 and 50° C.; it is a closed interval, i.e., comprising limits.

The pressure has little importance to the reaction, except if one of the reagents or the solvent have especially low boiling points.

For practical reasons, however, the pressure is preferably the atmospheric pressure of the location in question.

In the aforementioned case where one of the components of the reaction medium is especially volatile and where it is desired to keep this component in the reaction medium, it is then possible to increase the pressure; this pressure is generally then an autogenous pressure resulting from the reaction in a closed chamber.

Advantageously, the vinyl ester has the following formula (II):

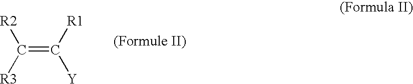

Another object of this invention is to provide a medium that can be used for carrying out electrolysis and that leads to heterocouplings. This object has been achieved by means of a composition that comprises at least:

a cobalt salt,

a conductive solvent or a solvent that was made conductive, and

a cobalt coordinating agent,

a vinyl ester.

The solvent and the coordinating agent of the cobalt can constitute one and the same entity, and even a single compound when the solvent is a single compound.

The cobalt content is advantageously between 2.10⁻³ and 10M, preferably between 5.10⁻³ and 5.10⁻² M (closed interval, i.e., including the limits). When cobalt-soluble anodes are used, the upper limiting values can be exceeded.

Said composition also comprises an aryl halide, whose preferred chemical characteristics will be presented in detail below. This aryl halide is advantageously present at a concentration of 0.1 to 1 M.

It is desirable that the molar ratio (dissolved radicals) of cobalt to vinyl ester go from 10⁻² to ½, preferably from 0.05 to 0.2 (closed interval, i.e., including the limits). The important limit values are the minimum values. If a cobalt-soluble anode is used, these values can be exceeded.

It is also sensible that the molar ratio (of course, radicals) of vinyl ester to aryl halide be at least equal to 1 and advantageously 1.5, preferably 2 and at most equal to 5, advantageously 4, and preferably 3. Thus, it is usually suitable that this ratio go from 1 to 5 (closed interval, i.e., including the limits). One skilled in the art will optimize this parameter, especially based on the nature of Y and the aromatic compound with which the vinyl is to be condensed.

The aryl substrates that can be coupled with the vinyls according to this invention represent a wide range of compounds. The halides are generally halides corresponding to relatively heavy halogens, i.e., halogens that are heavier than fluorine; these substrates are noted by formula (I):

Ar—X (Formula I)

It can also be indicated that when the halogen is linked to an aromatic core that is low in electrons, it is preferable to use bromines or chlorines as halogen, the chlorines being reserved for cores that are especially low in electrons. The condition is almost always met by heterocyclic compounds with six chain links, but in the case of homocyclic aryl hexacyclic substrates, to use a chloride, it is preferable that the sum of the Hammett constants ₚ of the substituents (not taking into account the starting halide) is at least equal to 0.4, preferably 0.5. By contrast, the cores that are especially high in electrons can be used as a halide or iodine.

For more details on Hammett constants, it is possible, for example, to refer to the third edition of the manual written by Professor Jerry March “Advanced Organic Chemistry” (pages 242 to 250) and edited by John Wiley and Sons.

The heterocyclic compounds with five chain links and that comprise as heteroatom a chalcogen (such as furan and thiophene) also yield acceptable results.

As was mentioned above, the reduction in electrons from the core can be caused either by the presence of electroattractor groups as substitutents, or, in the case of cores with six chain links, by the replacement of a carbon by a heteroatom. In other words, the core that is reduced in electrons can be a heterocyclic core with six chain links, especially the heterocyclic cores that have an atom from the column of nitrogen and more particularly the nitrogen.

Among the electroattractor groups leading to good results, it is suitable to cite acyl groups, nitrile groups, sulfone groups, carboxylate groups, trifluoromethyl groups or more generally perfluoroalkyl groups and halogens of a lower order than halide, which will be replaced by the vinyl radical.

Among the donor groups, i.e., yielding mediocre results with chlorine, but good results with bromine, it is possible to cite alkyloxyl groups, alkyl groups, amine groups and dialkylamine groups.

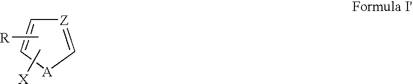

The aromatic derivative substrate of this process advantageously corresponds to the following formula, where:

Z represents a trivalent chain link —C(R₁′)=, and an atom of column V, advantageously a nitrogen;

X represents the starting halogen;

A represents either a link that is selected from among the ZH groups or from among the chalcogens that are advantageously of an order that is at least equal to that of sulfur, or from among the unsaturated divalent groups with two chain links

CR₂′=CR₃′, N═CR₂′CR₂=N.

To the extent that they are carried by contiguous atoms, two of the radicals R, R₁, R₂, and R₃ can be linked to form rings.

Substituents R, R₁, R₂′, and R₃′ are selected independently from among hydrogen and the substituents that are mentioned above in the passages that relate to AR—X. In particular, they can be selected from among the donor groups, among which it is possible to cite in particular the alkylchalcogenyl groups (such as alkyloxyl and the sulfur-containing derivative, and even the corresponding selenium), the alkyl groups, the amine groups, and dialkylamines.

Among the groups that have a relatively weak action on the electronic richness of the core that is being considered, in particular among the aryl groups, the acylchalcogenyl groups (such as acyloxyls [—CO—O-] and the sulfur-containing derivatives [—CO—S], and even corresponding selenia [—CO—Se]) and the halogens (the latter being preferably periodic and therefore with an atomic mass that is lower than halide, which will be replaced by the vinyl radical).

Among the strong electroattractor groups [i.e., whose Hammett constant up (sigma p) is at least equal to 0.4], especially the acyl groups, the nitrile groups, the sulfone groups, the carboxylate groups, the trifluoromethyl groups or more generally the perfluoroalkyl groups, and even the quaternary ammonium and the nitro group (the latter have a tendency to cause parasitic reactions, however).

Thus, the aryl compounds can be selected especially from among those of the following formulas:

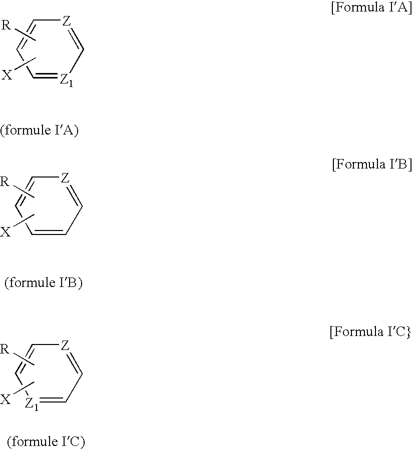

where:

Z₁ is selected from among the same meanings as those provided for Z;

radicals R₁, R₂, and R₃ are selected from among the aforementioned substituents and especially:

electroattractor groups, in particular acyl groups, nitrile groups, sulfone groups, carboxylate groups, trifluoromethyl groups, or more generally perfluoroalkyl groups and halogens of a lower order than halide that will be transformed into a coupling product;

donor groups, especially the aryloxyl groups, alkyloxyl groups, the hydrocarbyl groups such as aryl and alkyl (the latter word being used in its etymological meaning), amine groups, including groups that are mono- and disubstituted by alkylamine hydrocarbon groups.

It is desirable that the substrates have at most 50 carbon atoms, advantageously at most 30 carbon atoms, and preferably at most 20 carbon atoms.

The especially advantageous substrates include the halides, preferably aryl chlorides, that carry in particular in meta-position an aliphatic carbon (i.e., sp³) that carries at least two fluorines, for example halides, preferably trifluoromethylaryl chlorides.

One of the advantages of this invention is to require only complexing agents or coordinating agents, with easy access, such as nitriles (preferably aromatic or bidentate) or else the pyridines and the derivatives of the pyridine core, such as quinoline. Furthermore, the bipyridyls, being bidentate, also yield good results as a separate coordinating agent of the solvent.

This description now pertains to the implementation of heterocoupling between an aryl (pseudo)halide and an ester and even an ether of allyl that consists in subjecting the two substrates to a cathodic reduction in the presence of cobalt (II).

The reaction can be written roughly in the manner below:

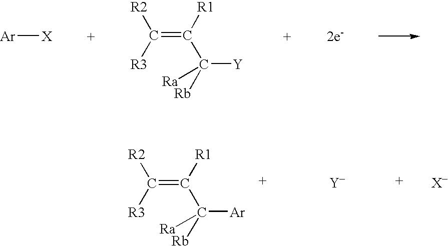

a formula in which Y corresponds to a leaving group that can exist in Y⁻ form, such as phenate, and even alcoholate, but advantageously selected from among the halogens and the carboxylates lato sensu and the pseudohalogens.

Ra and Rb, which can be identical or different, are selected from among the hydrocarbyls (i.e., the groups whose open bond is brought by a carbon and that comprises both hydrogen and oxygen) and hydrogens. It is desirable for preventing steric occupancy problems that at least one, preferably two, of the Ra and Rb is hydrogen.

Pseudohalogen is defined as designating a group whose removal leads to an oxidized anion, whereby the anionic charge is carried by the chalcogen atom, whose acidity is most often at least equal to that of the acetic acid, advantageously to the second acidity of the sulfuric acid and preferably to that of the trifluoroacetic acid. To be located on the scale of acidities, it is suitable to refer to the pKa for the middle to high acidities from the carboxylic acids to the trifluoroacetic acid and to be located on the scale of Hammett constants starting from the trifluoroacetic acid.

Carboxylate lato sensu should be defined as any radical such that its anionic form comprises the atomic sequence —CO—O —; thus, not only the carboxylate functions that are linked to a carbon atom but also the carbamic acids and the alkylcarbonates are targeted.

If it is desired to avoid all parasitic reactions, it is preferable to avoid the substituents comprising reactive hydrogens such as hydrogens on the amides (that are therefore advantageously protected or peralkylated) or on an oxygen.

Formulas in which R., R₂ and R₃, which may or may not be different, are selected from among hydrogen, the functions that are more difficult to reduce than the function Y, and among the hydrocarbon radicals, sometimes designated in this application by the term “hydrocarbyls,” in particular alkyls and aryls; whereby the alkyls are used in the etymological meaning of alcohol from which was removed the OH function, and comprises, of course, the aralkyls.

Thus, among the functions that are more difficult to reduce than Y, it is possible to cite the ether functions, the carboxylic functions, the functions among which Y is selected provided that these functions are less reducible than Y. The order of reducibility can be easily determined under the operating conditions by routine experiments. By way of indication, it can be noted that regarding the halides, the higher the atomic number, the more the halide is reducible, and in a more general (and rougher) way, the stronger the acid is which corresponds to the leaving group, the more the corresponding allyl ester is reducible (but it should be noted that the anions can themselves be reduced and cause parasitic reactions).

Among the groups that are relatively reducible and that should be emphasized, it is possible to cite the perfluorinated groups: one of the solutions is to influence the current density.

The hydrocarbon radicals are preferably either of an aromatic nature or an aliphatic nature, i.e., the carbon that ensures the link to the remainder of the molecule is hybridization carbon sp³; these aliphatic radicals are in general alkyls (alkyl is used in the etymological meaning of an alcohol from which the OH function is removed), including aralkyls. It should be pointed out that the hydrocarbon radicals that have a double bond conjugated with the allyl, the site of the reaction, yield only very mediocre results.

In general, the number of carbons from the allyl derivative is less than 50, advantageously 30.

Actually, during the studies that led to this invention, it was shown that in the presence of cobalt, the coupling above took place with good yields.

The reaction is actually an ipso reaction (here, the ablative of the Latin “ipse” is used to indicate that functionalization is done on the same carbon as the one that carried the starting halide or pseudohalide), but in some cases, of course when the allyl group is not palindrome, it was possible to observe small amounts of product corresponding to an SN′2.

Although the form of the cobalt in the vicinity of the metal in the elementary state had not been completely explained, it was possible to show that the presence of cobalt coordinating agents proved important and made it possible to increase the yields very significantly when they did not sequester very much. By contrast, the strong coordinating agents and primarily the strong bidentates are able to reduce the yield. Strong bidentate suitably means the bidentates of which one of the teeth is at least as complexing with regard to the cobalt as pyridine. The pyridine itself, when it is not engaged in a bidentate, yields excellent results. When reference is made to the bidentate concept, of course, the geometry of the molecule is defined as allowing two teeth to work together and therefore to form a ring with at most, advantageously less than, 7 centers with the cobalt.

Although an effect can be demonstrated when solvents that have the property of coordinating the cobalt are used, it is sometimes preferable to use specific coordinating agents.

If a return is made to the agents or solvation agents that make it possible to improve the yield significantly, it is possible to indicate that it is possible to use compounds that have a high donor index. More specifically, it is possible to indicate that it is preferable that donor index D of these solvents, or of these solvation agents, is greater than or equal to 10, preferably less than or equal to 30, advantageously between 20 and 30, including the limits. Said donor index corresponds to AH (enthalpy variation) that is expressed in kilocalories of the combination of said polar aprotic solvent or said coordinating agent with antimony pentachloride.

This is described more specifically in the work of Christian Reichardt: “Solvents and Solvent Effects in Organic Chemistry”—VCH, page 19, 1988. On this page is found the definition of the donor index that is expressed in English terms by “donor number.”

The results are better if the atom that coordinates the cobalt in said solvent or solvation agent is an atom from the column of nitrogen, and advantageously the nitrogen itself.

When a specific coordinating agent that does not play the role of solvent is used, it is possible to cite the functions or group of pyridine, nitrile, phosphine, stibine and imine.

To be effective, it is desirable that the cobalt be present at a minimum concentration of at least 10⁻³ M. Except in the case of strong bidentates, it is preferable that the ratio between the coordinating agents and the cobalt that is expressed in mol (coordinating agent(s)/Co) be at least equal to 1, advantageously 2, and preferably 5.

To be economical, it is preferable that the cobalt not be too concentrated; it is also preferred that the cobalt content be at most equal to 0.2 M.

The reaction medium advantageously comprises a solvent, and this solvent should be polar enough to dissolve the metals or more exactly the salts of the metals that are used, and it should be lipophilic enough to dissolve, at least partially, the substrates from which it is desired to form the allyl aryl.

It is preferable to use solvents that are low enough in acid so that the reactions with hydrogen are as little pronounced as possible. Thus, the primary alcohols are too acidic.

More specifically, the so-called polar aprotic solvents, such as the following, for example, alone or in a mixture, will be preferred:

Purely oxidized solvents, in particular ethers, preferably polyethers such as dimethoxy-1,2-ethane or cyclic ethers such as THF or dioxane;

amides or ureas (DMF, N-methylpyrrolidone-2, imidazolidone, tetramethyl urea, dimethoxypropylene-urea, etc.);

sulfones (for example, sulfolane) or sulfoxides (such as DMSO);

and, to the extent that they are liquid under the operating conditions, nitrogen-containing derivatives, nitrogen-containing heterocyclic compounds, especially pyridine, and compounds with a nitrile function (for those that are preferred, see below);

and, to the extent that they are liquid under the operating conditions, complexing agents (crown ether, HMPT) that improve the smooth running of the reaction by increasing conductivity, increasing the reactivity of the anion, and preventing metal deposits on the cathode.

Without this explanation being limiting, it would seem that these advantageous phenomena are correlated with the capacity to complex the metallic cations or in a mixture.

As indicated above, the solvents that are used can themselves play the part of complexing agents or coordinating agents. They can especially have one or more of the functions of coordination mentioned above.

The solvent can be a mixture of an apolar solvent and a polar solvent as defined above by the donor index.

When the solvent is not in itself a complexing agent of the cobalt that is strong enough to obtain optimum results, it is then desirable to use one of the complexing agents that is specific for cobalt, advantageously non-polydentate, and even non-bidentate, primarily when one of the teeth is a pyridine function. As functions that play the role of teeth, it is suitable to cite the nitrites (preferably aromatic and/or bidentate) or else the pyridines and the derivatives of the pyridine core, such as the quinoline. Alone, the dinitriles yield very good results.

The bipyridyls, being bidentate, thus yield mediocre results as separate complexing agents of the solvent. It is preferable that the complexing agents of the bidentate cobalt that comprise at least one pyridine as a tooth have a smaller amount than that of the cobalt (expressed in mol per liter).

More specifically, according to this invention, when the vinyl compounds are not treated, it is preferable that the complexing agents that are pyridinic in nature and that are expressed in terms equivalent to the pyridinic function or strong function be less than 2× the amount expressed in mol of cobalt salts, preferably less than 1×.

It is also desirable that the same rules apply to strong complexing agents of cobalt, such as the optionally bidentate amines and phosphines.

The preferred complexing agents are those that do not carry a charge, primarily negative, on the atom, or on the atoms that carry the bond coordinating the cobalt; it is also preferable that when said complexing agent carries a charge, the latter be located on the shortest path to at least 4, and even advantageously to at least 5 atoms, preferably 6, primarily when said charge is negative. Thus, the cyanides are not desirable as complexing agents of cobalt.

To facilitate the separation of products with the reaction media, it is preferable that said solvent exhibits a boiling point that is essentially different from the compound that is to be synthesized and the starting compound.

Said reaction can be conducted at many temperatures, but it is preferable to conduct this reaction at a temperature that is at most equal to 100 and at most equal to the boiling point of the solvent.

An interval yielding good results is the interval between 0 and 50° C.; it is a closed interval, i.e., including the limits.

Pressure is of little importance to the reaction, except if one of the reagents or the solvent has an especially low boiling point. For practical reasons, however, the pressure is preferably the atmospheric pressure of the location in question.

In the aforementioned case where one of the components of the reaction medium is especially volatile and where it is desired to keep this component in the reaction medium, it is then possible to increase the pressure; this pressure is generally then an autogenous pressure resulting from the reaction in a closed chamber.

Another object of this invention is to provide a medium that can be used for carrying out the reaction (probably an electrolytic microbattery) and that leads to heterocouplings. This object has been achieved by means of a composition that comprises at least:

a cobalt salt,

an optionally conductive solvent or a solvent that was made conductive, and

a cobalt coordinating agent,

an ester or an ether of allyl, and even of homoallyl.

The solvent and the coordinating agent of the cobalt can constitute one and the same entity, and even a single compound when the solvent is a single compound.

The cobalt content is advantageously between 2.10⁻³ and 10⁻¹ M, preferably between 5.10⁻³ and 5.10⁻² M (closed interval, i.e., including the limits). When cobalt-soluble anodes are used, the upper limiting values can be exceeded.

The cobalt can be potentialized by iron (II). The latter can be present at a level such that the Co/Fe ratio goes from 1/10 to 10/1, advantageously from 1/5 to 5/1.

This potentialization is particularly advantageous when an aryl chloride is coupled to an ester or an ether of allyl, and even of homoallyl.

Said composition also comprises an aryl halide (Ar—X) whose preferred chemical characteristics will be presented in detail below. This aryl halide is advantageously present at a concentration of at least 0.01 M, preferably 0.1 to 1 M.

It is desirable that the allyl ester or allyl ether be at a concentration (dissolved) of 0.01 M.

It is desirable that the molar ratio (dissolved radicals) of cobalt to allyl ester goes from 10⁻² to ½, preferably from 0.05 to 0.2 (closed interval, i.e., including the limits). The significant limit values are the minimum values. In the case of a cobalt-soluble anode being used, these values can be exceeded.

It is also sensible that the molar ratio (of course, radicals) of allyl ester or allyl ether to aryl halide be at least equal to 1 and advantageously 1.5, preferably 2 and at most equal to 5, advantageously 4, and preferably 3. Thus, it is usually suitable that this ratio go from 1 to 5 (closed interval, i.e., including the limits). One skilled in the art will optimize this parameter, especially based on the nature of Y and the aromatic compound with which the allyl is to be condensed.

According to an implementation of the invention, the intensity and the surface of the reactive electrode, more exactly of the electrode where the reaction-takes place, such that the density of current j is advantageously at least equal to between 5 and advantageously at most equal to 5.10² A/m², preferably between 20 and 200 A/m² (closed interval, i.e., including the limits). In the case of microbatteries, however, the measurement and the control of the intensity cannot be done easily. The values show primarily that the area is broad. It is always possible, however, to act on the potential of the alloy or the metal or on its specific surface (factors that influence the current density) to optimize the selectivity and the yield without thereby measuring the current density.

By routine tests, one skilled in the art can determine the potential for cobalt reduction in the reaction medium and that of aryl halide. This determination made, it will preferably be placed between the cobalt reduction potential and that of the aryl halide. Within the scope of this invention, this potential is the potential for dissolution, in the reaction medium, of the alloy or the metal that is used as a reducing agent.

In the case of allyl derivatives, the zinc yields good results especially in the heterocoupling with the heavy aryl halides (bromides or iodides).

The aryl substrates (Ar—X) that can be coupled to the allyls according to this invention represent a wide range of compounds. The halides are generally halides corresponding to relatively heavy halogens, i.e., halogens that are heavier than fluorine.

It can also be indicated that when the halogen is linked to an aromatic core that is low in electrons, it is preferable to use bromines or chlorines as halogen, the chlorines being reserved for cores that are especially low in electrons. The condition is almost always met by heterocyclic compounds with six chain links, but in the case of homocyclic aryl hexacyclic substrates, to use a chloride, it is preferable that the sum of the Hammett constants p of the substituents (not taking into account the starting halide) be at least equal to 0.4, preferably 0.5. By contrast, the cores that are especially high in electrons can be used as a halide or iodine.

For more details on Hammett constants, it is possible, for example, to refer to the third edition of the manual written by Professor Jerry March “Advanced Organic Chemistry” (pages 242 to 250) and edited by John Wiley and Sons.

The heterocyclic compounds with five chain links and that comprise as heteroatom a chalcogen (such as furan and thiophene) also yield acceptable results.

As has been mentioned above, the reduction in electrons from the core can be caused either by the presence of electroattractor groups as substitutents, or, in the case of cores with six chain links, by the replacement of a carbon by a heteroatom. In other words, the core that is reduced in electrons can be a heterocyclic core with six chain links, especially the heterocyclic cores that have an atom from the column of nitrogen and more particularly the nitrogen.

Among the electroattractor groups leading to good results, it is suitable to cite acyl groups, nitrile groups, sulfone groups, carboxylate groups, trifluoromethyl groups or more generally perfluoroalkyl groups and halogens of a lower order than halide, which will be replaced by the allyl radical.

Among the donor groups, i.e., yielding mediocre results with chlorine, but good results with bromine, it is possible to cite alkyloxyl groups, alkyl groups, amine groups and dialkylamine groups.

The aromatic derivative substrate of this process corresponds advantageously to the following formula:

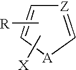

where:

Z represents a trivalent chain link (R)═, and an atom of column V, advantageously a nitrogen;

X represents a starting halogen (and even a starting pseudohalogen);

A represents either a link that is selected from among the ZH groups or from among the chalcogens that are advantageously of an order that is at least equal to that of sulfur, or from among the unsaturated divalent groups with two chain links

C R₂═CR₃N═CR₂CR₂═N.

To the extent that they are carried by the contiguous atoms, two of the radicals R, R₁, R₂, and R₃ can be linked to form rings.

Thus, the aryls can have especially the formulas:

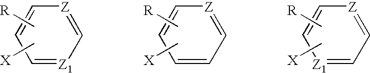

where:

Z₁ is selected from among the same meanings as those provided for Z;

radicals R, R₁′, R₂′, and R₃′ are selected from among the aforementioned substituents and especially:

electroattractor groups, in particular acyl groups, nitrile groups, sulfone groups, carboxylate groups, trifluoromethyl groups or more generally perfluoroalkyl groups and halogens of a lower order than halide that will be transformed into a coupling product;

donor groups, especially the aryloxyl groups, alkyloxyl groups, the hydrocarbyl groups such as aryl and alkyl (the latter word being used in its etymological meaning), amine groups, including groups that are mono- and disubstituted by alkylamine hydrocarbon groups.

It is desirable that the substrates have at most 50 carbon atoms, advantageously at most 30 carbon atoms, and preferably at most 20 carbon atoms.

The especially advantageous substrates include the halides, preferably aryl chlorides, that carry in particular in meta-position an aliphatic carbon (i.e., Sp³) that carries at least two fluorines, for example halides, preferably trifluoromethylaryl chlorides.

One of the advantages of this invention is to require only complexing agents or coordinating agents, with easy access, such as nitrites (preferably aromatic or bidentate) or else the pyridines and the derivatives of the pyridine core, such as quinoline. Furthermore, the bipyridyls, being bidentate, also yield good results as a separate coordinating agent of the solvent.

The FG or GF of the formulas of the examples constitute examples of values of R in the developed formulas of Ar—X.

The following non-limiting examples illustrate the invention.

### EXAMPLES THAT PERTAIN TO ARYL-ALLYL COUPLING

Operating procedure of the chemical vinylation of aromatic chlorides:

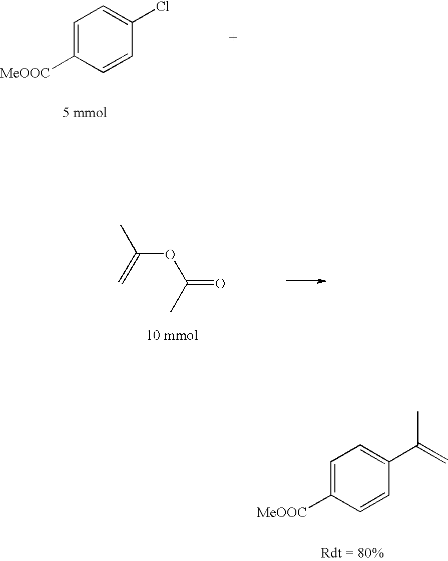

Solvents: DMF (15 ml)+pyridine (2 ml)

MeOCO-Ph-Cl: 5 millimol

Isopropenyl acetate: 10 millimol

CoBr₂: 0.5 millimol (10%/ArCl)

2,2′-bipyridine: 0.5 millimol (10%/ArCl)

Manganese powder: 50 millimol (this amount can be decreased)

0.1 ml of CF₃COOH

Temperature: 50° C.

Reaction time: 1 hour 30 minutes to 2 hours

### Example 2

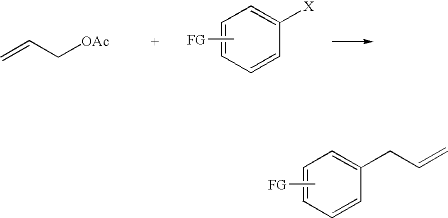

i:X=Br: MeCN, CoBr₂ cata, Zn, RT

FG means a functional group (i.e., a substituent) that is not touched during the reaction.

An acetonitrile solution was formed by successive addition to 20 ml of acetonitrile of a zinc powder (3.25 g, 50 mmol), cobalt bromide CoBr₂ (0.657 g, 3 mmol), zinc bromide ZnBr₂ (0.338 g, 1.5 mmol), and bromobenzene (0.16 g, 1.5 mmol). The reagent is activated by an addition of acetic acid (0.024 g, 4.10⁻⁴ mmol).

It is then stirred for 30 minutes at ambient temperature (23° C.); there is a disappearance of bromobenzene. Allyl acetate (3.03 g, 30 mmol) and ethyl p-bromobenzoate (2.427 g, 5 mmol) are then introduced into the solution after stirring for 3 hours at ambient temperature. The reaction mixture is thrown into a solution of 2 M HCl (40 ml), and the whole is subjected to a liquid-liquid extraction by means of ethyl ether (3×40 ml). The organic phase is dried on evaporated MgSO₄ and finally purified on a silica gel chromography column (pentane/ether eluant, 99/1). It yields 1.852 g (65%) of ethyl p-(2-propenyl) benzoate in the form of colorless oil.

The reaction is carried out in the same way on different allyl bromides.

TABLE 1
Allylation of Aryl Bromides by Allyl Acetate with the Co—Zn Pair

| | Sub- | | | |
|---|---|---|---|---|
| | stituent | | | Other |
| | of Bromo- | | Yield,^{a} | Pro- |
| Test | benzene | Product | % | ducts |
| 1 | p-COOEt | 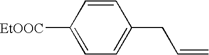 | 65 | ArH 20% ArAr 15% |
| 2 | p-CN | 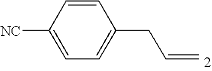 | 75 | ArH 25% |
| 3 | p-CF₃ | 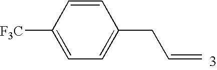 | 50 | ArH 40% ArAr 10% |
| 4 | p-OMe | 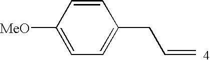 | 60 | ArH 27% ArAr 13% |
| 5 | m-COOEt | 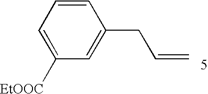 | 51 | ArH 36% ArAr 13% |
| 6 | m-CF₃ | 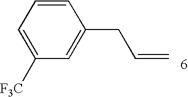 | 31 | ArH 54% ArAr 15% |
| 7 | m-Cl | 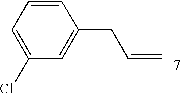 | 54 | ArH 34% ArAr 12% |
| 8 | o-CN | 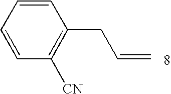 | 66 | ArH 29% ArAr 5% |
| 9^{‡} | H | 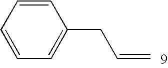 | 35 | ArH 50% ArAr 15% |

^{a}yield of pure isolated product
^{‡}Here, the bromobenzene is used both as an activating agent and as a substrate.

### Example 3

### Allylation of Aryl Chloride

Allyl acetate (10 mmol) and aryl chloride (5 mmol) are introduced into a pyridine acetonitrile solution (AN=20 ml/Py=2 ml) that contains manganese dust (50 mmol), cobalt bromide dust (2 mmol), and ferrous bromide dust (5 mmol). The reaction mixture is brought to 50° C. and is stirred until aryl chloride completely disappears (not detectable in gas chromatography).

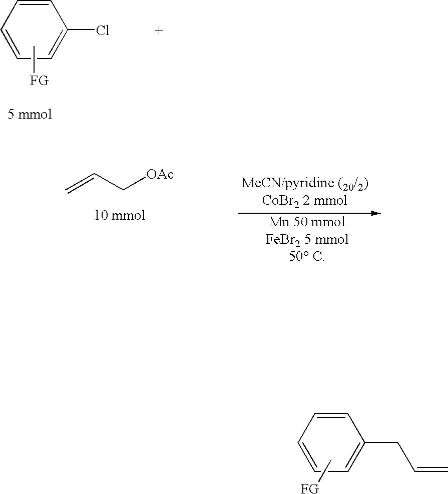

TABLE 2
Allylation of Aryl Chlorides by Allyl Acetate (with Co—Fe—Zn)

| | Substituent | | | |
|---|---|---|---|---|
| Test | of Chlorobenzene | Product | Yield,^{a} (%) | Other Products |
| 10 | p-COOMe | 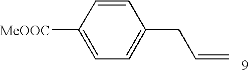 | 72 | ArH 10% ArAr 18% |
| 11 | p-CN | 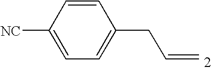 | 83 | ArH 17% |
| 12 | o-CN | 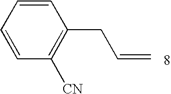 | 83 | ArH 14% ArAr 3% |
| 13 | p-CF₃ | 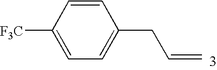 | 50 | ArH 26% ArAr 24% |
| 14 | H | 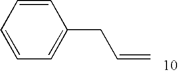 | 14^{b} | ArH 48% ArAr 38% |

^{a}Yield of pure isolated product.
^{b}Yield measured in gas chromatography.

### Example 4

### Synthesis of Para-acetyloxystyrene

Reaction:

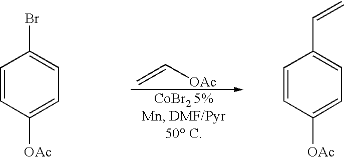

Operating Procedure:

In a 200 ml glass reactor under argon, the following are introduced successively: 4.30 g (50 mmol) of vinyl acetate, 75 ml of dry DMF, 5.37 g (25 mmol) of p-Br—Ph-OAc, 13.750 g (250 mmol) of Mn, 10 ml of pyridine, 0.273 g (1.25 mmol) of CoBr₂, 0.195 g (1.25 mmol) of 2,2′-bipyridine and 0.5 μl of CF₃COOH. This reaction mass is brought to 50° C. After 30 minutes, a gas chromatography analysis shows that the degree of transformation of p-Br—Ph-OAc is complete. It is allowed to cool to ambient temperature, and it is filtered on a mixture of black (4s) and Celite and taken up by 120 ml of ethyl acetate. The organic phase is washed with 60 ml of a 2N HCl solution. The aqueous phase is extracted 2× by 60 ml of ethyl acetate. The organic phases are collected and washed by 60 ml of an NaCl-saturated solution. 146 g of organic phase that is dried on MgSO₄ is obtained. After evaporation, 8 g of a colorless oil that contains for the most part the derivative p-AcOPh-Vi is obtained.

### Example 5

### Coupling of the Methyl-p-chlorobenzoate with the Cyclopentyl Acetate

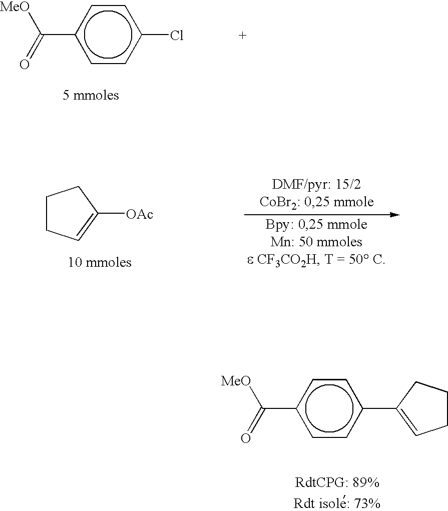

[Key to Example 5:]

mmoles=mmol

Rdt CPG=GC Yield

Rdt isolé=Isolated yield

### Example 6

### Coupling of ArCl with Vinyl Acetate

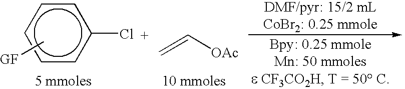

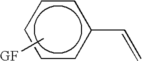

| | Functional | | | |
|---|---|---|---|---|
| Input | Group | Final Time | GC Yield (%) | Isolated Yield (%) |
| 1 | p-MeOCO | 2 hours 00 min | 82 | 81 |
| 2 | p-MeCO | 1 hour 30 min | 48 | 28 |

### Example 7

### Case of 3-Bromothiophene (Heterocyclic Compound with Five Chain Links, High in Electrons)

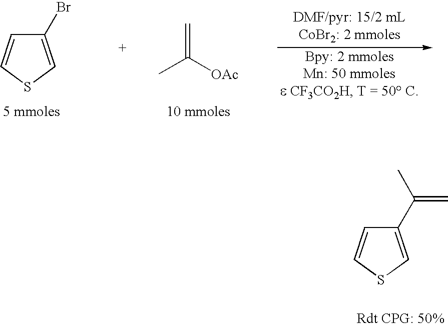

[Key to Example 7:]

mmoles=mmol

Rdt CPG=GC Yield

The heterocyclic compounds with five chain links are considered to be high in electrons; bromide was therefore used.

### Example 8

### Other Ar Br

Reaction:

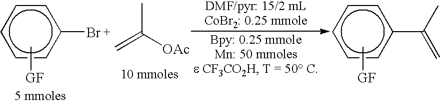

| Input | GF | Final Time | GC Yield (%) | Isolated Yield (%) |
|---|---|---|---|---|
| 1 | p-MeO | 3 hours 40 min | 84 | 78 |
| 2 | p-CF₃ | 3 hours 10 min | 60 | |
| 3 | p-AcO | 1 hour 30 min | 89 | 74 |

### Example 9

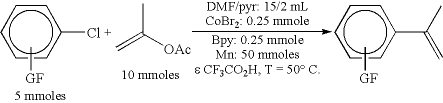

| In- | | | | |
|---|---|---|---|---|
| put | GF | Final Time | GC Yield (%) | Isolated Yield (%) |
| 1 | p-MeOCO | 3 hours 30 min | 86 | 75 |
| 2 | p-CN | 1 hour 30 min | 84 | 76 |
| 3 | p-CF₃ | 8 hours 00 min | 72 | 35 |
| 4 | o-CF₃ | 18 hours (night) | 15 | |
| 5 | p-MeCO | 2 hours 30 min | 80 | 65 |
| 6 | o-MeCO | 2 hours 30 min | 53 | |
| 7 | o-CN | 4 hours | 94 | 69 |

1. Use of cobalt as a catalyst for heterocoupling between an aryl (pseudo)halide and a derivative that carries a double bond and a leaving group in vinyl-position, allyl-position and even homoallyl-position of said double bond by being brought into contact in a solvent with a metal or an alloy that is at least as reducing as zinc.

2. Use according to claim 1, characterized by the fact that the cobalt is present in oxidation state 2.

3. Use according to claims 1 and 2, wherein the cobalt is present in a coordinated form.

4. Use according to claim 3, wherein the coordination of the cobalt is carried out by a solvent compound or solvating compound that has a high donor index.

5. Use according to claim 4, wherein the atom that is responsible for a good donor index is selected from among the atoms of the nitrogen column.

6. Use according to claims 3 to 5, wherein the coordination of the cobalt is carried out by a specific coordinating agent.

7. Use according to claim 6, wherein said coordinating agent has functions that are selected from among the pyridine, nitrile, phosphine, stibine and imine functions.

8. Use according to claims 1 to 7, wherein the metal is selected from among zinc and metals and alloys that are more electro-reducing than zinc.

9. Use according to claim 8, wherein the metal is selected from among manganese and metals that are at least as reducing as manganese, provided that when the metal is more electro-reducing than the manganese, the medium contains manganous ions.

10. Use according to claims 8 and 9, wherein said metal is selected from among the metals that are more electro-reducing than the manganese and wherein the medium contains manganous ions, advantageously at a concentration of between 2×10⁻³ and 10⁻¹ M.

11. Use according to claims 1 to 10, wherein said derivative that carries a double bond and a leaving group is a vinyl ester.

12. Use according to claim 11, wherein the ratio of (coordinating agent(s)/cobalt) between coordinating agent(s), expressed in mol for the monodentates and in equivalent terms for the polydentates and the cobalt ions (expressed in mol) is at least equal to 0.5; advantageously 1, preferably 2 and more preferably 4.

13. Use according to claims 1 to 12, wherein said derivative that carries a double bond and a leaving group is an allyl ester or an allyl ether.

14. Use according to claim 13, wherein the ratio of [cobalt]/[coordinating agent that is at least bidentate and of which at least one tooth is pyridine expressed in pyridine equivalent] is greater than ½, advantageously 1.

15. Use according to claim 14, wherein when the complexing agents are strong complexing agents, the cobalt/pyridine equivalent ratio is greater than ½, advantageously 1.

16. Composition that comprises at least one cobalt salt, a solvent that is optionally conductive or made conductive, a cobalt coordinating agent and a derivative that carries a double bond and a leaving group.

17. Composition according to claim 16, wherein it also comprises a ferrous salt, advantageously in dissolved form.

18. Composition according to claims 16 and 17, wherein it comprises a ferrous salt, the Co/Fe ratio from 1/10 to 10/1, advantageously from 1/5 to 5/1.

19. Composition according to claims 16 to 18, wherein the cobalt content is between 2×10⁻³ et 10⁻¹ M.

20. Composition according to claims 16 to 19, wherein it comprises a solvent that is selected from among the components below, alone or in a mixture:
Purely oxidized solvents, in particular the ethers, preferably polyethers such as dimethoxy-1,2-ethane or cyclic ethers such as THF or dioxane;
Amides, including ureas;
Sulfones or sulfoxides;
Nitrogen-containing derivatives, in particular nitrogen-containing heterocyclic compounds, in particular pyridine and compounds with nitrile functions;
Complexing agents.

21. Composition according to claims 16 to 20, wherein the molar ratio of dissolved radical between the cobalt and a derivative that carries a double bond and a leaving group goes from 10⁻² to 0.5.

22. Process of coupling an aryl (pseudo)halide with a derivative that carries a double bond and a leaving group, wherein it consists in subjecting a composition according to one of claims 16 to 19, also comprising an aryl (pseudo)halide to a reaction with a metal that is at least as electro-reducing as zinc.

23. Process according to claim 22, wherein the aryl (pseudo)halide is a compound of formula (I):
Ar—X (Formula I)
where X represents a halogen atom that is heavier than the fluorine, and
where Ar represents a homocyclic or heterocyclic aromatic radical.

24. Process according to claims 22 and 23, wherein the aryl (pseudo)halide is a compound of formula (I):
Ar—X (Formula I)
where X represents a bromine or chlorine atom, and
where Ar represents an aromatic radical whose core that carries X is stripped of electrons.

25. Process according to claims 22 to 24, wherein the aryl (pseudo)halide is a compound of formula (I):
Ar—X (Formula I)
where X represents a bromine or chlorine atom, and
where Ar represents an aromatic radical whose core that carries X is stripped of electrons and is selected from among the aromatic compounds of which said core carries electroattractor function(s) and/or group(s) and whose substituents are such that the sum of their Hammett constants cup (sigma p) is greater than zero.

26. Process according to claims 22 to 25, wherein the aryl (pseudo)halide is a compound of formula (I):
Ar—X (Formula I)
where X represents a chlorine atom, and
where Ar represents an aromatic radical whose core that carries X is stripped of electrons and is selected from among the aromatic compounds of which said core carries (an) electroattractor function(s) and/or group(s) and whose substituents are such that the sum of their Hammett constants up (sigma p) is at least equal to 0.4, preferably 0.5 or of which said core is a heterocyclic compound with 6 chain links that advantageously have an atom from the nitrogen column, and especially nitrogen and phosphorus.

27. Process according to claim 23, wherein the aryl (pseudo)halide is a compound of formula (I):
Ar—X (Formula I)
where X represents a bromine or iodine atom, and
where Ar represents an aromatic radical whose core that carries X is not stripped of electrons.

28. Process according to claims 22 to 27, wherein said derivative that carries a double bond is a vinyl ester of formula (II) 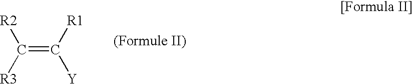
where R₁, R₂ et R₃, which may or may not be different, are selected from among hydrogen, the functions that are more difficult to reduce than the function Y and from among the hydrocarbon radicals, in particular alkyls and aryls;
where Y corresponds to a leaving group that can exist in the form Y—, advantageously selected from among the halogens and the carboxylates and
wherein said metal or alloy is more electro-reducing than the zinc.

29. Process according to claim 28, wherein the cobalt is complexed by a bidentate coordinating agent, advantageously of which one of the teeth is a pyridine, preferably of which the two teeth are pyridines, more preferably bipyridine.

30. Process according to claims 28 and 29, wherein said radical Ar is an acyloxyphenyl.

31. Process according to claims 28 and 30, wherein said vinyl acetate is selected from among the vinyl alcanoates per se (where R₁, R₂, and R₃ are H) and the isopropenyl alkanoates (one of R₁, R₂, and R₃ is methyl and the others are hydrogens).
